(19) United States
(12) Patent Application Publication
(10) Pub. No.: US 2018/0221254 A1
(43) Pub. Date: Aug. 9, 2018

(54) COMPOSITIONS CONTAINING LATEX PARTICLES AND INORGANIC METAL OXIDES

(71) Applicant: Rohm and Haas Company, Collegeville, PA (US)

(72) Inventors: Junsi Gu, Malvern, PA (US); Jennifer Koenig, Lansdale, PA (US); Xiang Qian Liu, Collegeville, PA (US); Xiaodong Lu, North Wales, PA (US); Ying O'Connor, Coatesville, PA (US); Curtis Schwartz, Ambler, PA (US); Xindi Yu, Phoenixville, PA (US); Fanwen Zeng, Audubon, PA (US)

(73) Assignee: Rohm and Haas Company, Collegeville, PA (US)

(21) Appl. No.: 15/749,317

(22) PCT Filed: Aug. 3, 2016
(86) PCT No.: PCT/US16/45321

§ 371 Date: Jan. 31, 2018

Related U.S. Application Data
(60) Provisional application No. 62/366,336, filed on Jul. 25, 2016.
(60) Provisional application No. 62/202,193, filed on Aug. 7, 2015.

(51) Int. Cl. A61K 8/02; A61K 8/27; A61K 8/29; A61K 8/81; A61Q 19/02

(52) U.S. Cl. CPC A61K 8/0279; A61K 8/27; A61K 8/29; A61K 2800/412; A61K 8/8129; A61K 8/8152; A61Q 19/02; A61K 8/8117

(57) ABSTRACT

Provided are personal care compositions comprising (A) voided latex particles comprising (i) at least one core polymer comprising polymerized units derived from monoethylenically unsaturated monomers containing at least one carboxylic acid group, and non-ionic ethylenically unsaturated monomers, and (ii) at least one shell polymer comprising polymerized units derived from non-ionic ethylenically unsaturated monomers, and polyethylenically unsaturated monomers, and (B) pigment grade inorganic metal oxide particles, wherein the voided latex particles contain a void and have a particle size of from 400 nm to 800 nm. Also provided are methods for lightening skin tone comprising topically administering such compositions to the skin, and methods for improving visible light scattering of a composition including a pigment grade inorganic metal oxide particle comprising adding to such composition a voided latex particle.

### FIELD OF THE INVENTION

This invention relates generally to personal care compositions comprising voided latex particles and inorganic metal oxide particles.

### BACKGROUND

Personal care compositions contain a variety of additives that provide a wide array of benefits to users, such as treating skin to alter its color or tone, for example, by skin lightening. Pigment grade inorganic metal oxide particulate light scatterers, such as zinc oxide and titanium dioxide, are effective skin lightening agents. Providing long lasting whitening appearance is a challenging goal to meet, however. Most current technology, for example, are effective for providing whitening appearance for less than an hour.

Personal care compositions comprising light scatterers and UV absorbing agents have been disclosed. For example, U.S. Pat. No. 5,663,213 discloses a method of improving UV radiation absorption of a composition containing at least one UV radiation absorbing agent by incorporating a voided latex particle into the composition. Although the prior art discloses such particles for use in boosting the UV absorption of a composition in combination with a UV absorbing agent, the prior art does not disclose a voided latex particle with a particle size that is effective for skin lightening with a light scattering agent useful in skin lightening applications.

Consequently, there is a need to develop new personal care compositions for use in skin lightening applications, including compositions that improve upon the state of the art with respect to the effectiveness of such compositions over time.

### STATEMENT OF INVENTION

One aspect of the invention provides a personal care composition comprising (A) voided latex particles comprising (i) at least one core polymer comprising polymerized units derived from (a) 20 to 60 weight % of monoethylenically unsaturated monomers containing at least one carboxylic acid group, based on the total weight of the core polymer, and (b) 40 to 80 weight % of non-ionic ethylenically unsaturated monomers, based on the total weight of the core polymer, and (ii) at least one shell polymer comprising polymerized units derived from (a) 55 to 85 weight % of non-ionic ethylenically unsaturated monomers, based on the total weight of the shell polymer(s), and (b) 15 to 45 weight % of polyethylenically unsaturated monomers, based on the total weight of the shell polymer(s) and (B) at least one pigment grade inorganic metal oxide particle, wherein the voided latex particles are present in an amount of from 0.5 to 10 weight %, based on the total weight of the composition, and wherein the voided latex particles contain a void and have a particle size of from 400 nm to 1500 nm.

Another aspect of the invention provides a method for lightening skin tone, comprising topically administering to the skin an effective amount of a personal care composition comprising (A) voided latex particles comprising (i) at least one core polymer comprising polymerized units derived from (a) 20 to 60 weight % of monoethylenically unsaturated monomers containing at least one carboxylic acid group, based on the total weight of the core polymer, and (b) 40 to 80 weight % of non-ionic ethylenically unsaturated monomers, based on the total weight of the core polymer, and (ii) at least one shell polymer comprising polymerized units derived from (a) 55 to 85 weight % of non-ionic ethylenically unsaturated monomers, based on the total weight of the shell polymer(s), and (b) 15 to 45 weight % of polyethylenically unsaturated monomers, based on the total weight of the shell polymer(s), and (B) pigment grade inorganic metal oxide particles, wherein the voided latex particles are present in an amount of from 0.5 to 10 weight %, based on the total weight of the composition, and wherein the voided latex particles contain a void and have a particle size of from 400 nm to 1500 nm.

In another aspect, the invention provides a method for improving visible light scattering of a composition comprising adding to said composition from 0.5 to 10 weight % of voided latex particles, based on the total weight of the composition, comprising (i) at least one core polymer comprising polymerized units derived from (a) 20 to 60 weight % of monoethylenically unsaturated monomers containing at least one carboxylic acid group, based on the total weight of the core polymer, and (b) 40 to 80 weight % of non-ionic ethylenically unsaturated monomers, based on the total weight of the core polymer, and (ii) at least one shell polymer comprising polymerized units derived from (a) 55 to 85 weight % of non-ionic ethylenically unsaturated monomers, based on the total weight of the shell polymer(s), and (b) 15 to 45 weight % of polyethylenically unsaturated monomers, based on the total weight of the shell polymer(s), wherein the composition comprises at least one pigment grade inorganic metal oxide particle, and wherein the voided latex particles contain a void and have a particle size of from 400 nm to 1500 nm.

### BRIEF DESCRIPTION OF THE FIGURES

FIG. 1 shows the reflectance of a control film (C1), comparative film including 1 weight % TiO2 (C2), exemplary film including 1 weight % TiO₂+1 weight % 400 nm voided latex particles (E1), and exemplary film including 1 weight % TiO2+2 weight % 400 nm voided latex particles (E2).

FIG. 2 shows the reflectance of a control film (C1), comparative film including 2 weight % TiO2 (C3), comparative film including 2 weight % 400 nm voided latex particles (C4), exemplary film including 2 weight % TiO₂+1 weight % 400 nm voided latex particles (E3), and exemplary film including 2 weight % TiO₂+2 weight % 400 nm voided latex particles (E4).

FIG. 3 shows the reflectance of a comparative film including 1 weight % TiO2+1 weight % 350 nm voided latex particles (C5), exemplary film including 1 weight % TiO₂+1 weight % 400 nm voided latex particles (E1), and exemplary film including 1 weight % TiO₂+1 weight % 550 nm voided latex particles (E5).

FIG. 4 shows the reflectance of a comparative film including 2 weight % TiO₂+2 weight % 350 nm voided latex particles (C6), exemplary film including 2 weight % TiO₂+2 weight % 400 nm voided latex particles (E4), and exemplary film including 2 weight % TiO₂+2 weight % 550 nm voided latex particles (E6).

FIG. 5 shows the reflectance of a comparative film including 2 weight % TiO₂ (C3) and exemplary film including 1 weight % TiO2+1 weigh % 1000 nm voided latex particles (E7).

FIG. 6 shows the reflectance of a comparative film including 4 weight % TiO₂ (C7) and exemplary film including 2 weight % TiO₂+2 weigh % 1000 nm voided latex particles (E8).

### DETAILED DESCRIPTION

The inventors have now surprisingly found that voided latex particles comprising a core polymer and a shell polymer, and having a particle size of from 400 nm to 1500 nm provide improved light scattering to compositions containing pigment grade inorganic metal oxide particles, as well as providing a long lasting whitening effect when applied to skin. Accordingly, the present invention provides in one aspect a personal care composition comprising voided latex particles and light scattering pigment grade inorganic metal oxide particles.

In the present invention, “personal care” is intended to refer to cosmetic and skin care compositions for leave on application to the skin including, for example, lotions, creams, gels, gel creams, serums, toners, wipes, masks, liquid foundations, make-ups, tinted moisturizer, oils, face/body sprays, and topical medicines, as well as rinse off application to the skin including, for example, body/face/hand washes, soaps, and cleansers. “Personal care” relates to compositions to be topically administered (i.e., not ingested). Preferably, the personal care composition is cosmetically acceptable. “Cosmetically acceptable” refers to ingredients typically used in personal care compositions, and is intended to underscore that materials that are toxic when present in the amounts typically found in personal care compositions are not contemplated as part of the present invention. The compositions of the invention may be manufactured by processes well known in the art, for example, by means of conventional mixing, dissolving, granulating, emulsifying, encapsulating, entrapping or lyophilizing processes.

As used herein, the term “polymer” refers to a polymeric compound prepared by polymerizing monomers, whether of the same or a different type. The generic term “polymer” includes the terms “homopolymer,” “copolymer,” and “terpolymer.” As used herein, the term “polymerized units derived from” refers to polymer molecules that are synthesized according to polymerization techniques wherein a product polymer contains “polymerized units derived from” the constituent monomers which are the starting materials for the polymerization reactions. As used herein, the term “(meth)acrylic” refers to either acrylic or methacrylic.

As used herein, the terms “glass transition temperature” or “T_{g}” refers to the temperature at or above which a glassy polymer will undergo segmental motion of the polymer chain. Glass transition temperatures of a polymer can be estimated by the Fox equation (Bulletin of the American Physical Society, 1 (3) Page 123 (1956)) as follows:

1/T_{g}=w₁/T_{g(1)}+w₂/T_{g(2)}

For a copolymer, w₁ and w₂ refer to the weight fraction of the two comonomers, and T_{g(1)} and T_{g(2)} refer to the glass transition temperatures of the two corresponding homopolymers made from the monomers. For polymers containing three or more monomers, additional terms are added (wₙ/T_{g(n)}). The T_{(g)} of a polymer can also be calculated by using appropriate values for the glass transition temperatures of homopolymers, which may be found, for example, in “Polymer Handbook,” edited by J. Brandrup and E. H. Immergut, Interscience Publishers. The T_{g} of a polymer can also be measured by various techniques, including, for example, differential scanning calorimetry (“DSC”). The values of T_{g} reported herein are measured by DSC.

The inventive personal care compositions contain voided latex particles. Voided latex particles useful in the invention comprise a multistaged particle containing at least one core polymer and at least one shell polymer. The ratio of the core weight to the total polymer weight is from 1:4 (25% core) to 1:100 (1% core), and preferably from 1:8 (12% core) to 1:50 (2% core).

The at least one core polymer includes polymerized units derived from monoethylenically unsaturated monomers containing at least one carboxylic acid group, and non-ionic ethylenically unsaturated monomers. The core polymer may be obtained, for example, by the emulsion homopolymerization of the monoethylenically unsaturated monomer containing at least one carboxylic acid group or by copolymerization of two or more of the monoethylenically unsaturated monomers containing at least one carboxylic acid group. In certain embodiments, the monoethylenically unsaturated monomer containing at least one carboxylic acid group is copolymerized with one or more non-ionic (that is, having no ionizable group) ethylenically unsaturated monomers. While not wishing to be bound by theory, it is believed that the presence of the ionizable acid group makes the core swellable by the action of a swelling agent, such as an aqueous or gaseous medium containing a base to partially neutralize the acid core polymer and cause swelling by hydration.

Suitable monoethylenically unsaturated monomers containing at least one carboxylic acid group of the core polymer include, for example, (meth)acrylic acid, (meth)acryloxypropionic acid, itaconic acid, aconitic acid, maleic acid, fumaric acid, crotonic acid, citraconic acid, maleic anhydride, monomethyl maleate, monomethyl fumarate, and monomethyl itaconate, and other derivatives such as corresponding anhydride, amides, and esters. In certain preferred embodiments, the monoethylenically unsaturated monomers containing at least one carboxylic acid group are selected from acrylic acid and methacrylic acid. In certain embodiments, the core comprises polymerized units of monoethylenically unsaturated monomers containing at least one carboxylic acid group in an amount of from 20 to 60 weight %, preferably from 30 to 50 weight %, and more preferably from 35 to 45 weight %, based on the total weight of the core polymer.

Suitable non-ionic ethylenically unsaturated monomers of the core polymer include, for example, styrene, vinyltoluene, ethylene, vinyl acetate, vinyl chloride, vinylidene chloride, acrylonitrile, (meth)acrylamide, (C₁-C₂₂)alkyl and (C₃-C₂₀)alkenyl esters of (meth)acrylic acid, such as methyl (meth)acrylate, ethyl (meth)acrylate, butyl (meth)acrylate, 2-ethylhexyl (meth)acrylate, benZyl (meth)acrylate, lauryl (meth)acrylate, oleyl (meth)acrylate, palmityl (meth)acrylate and stearyl (meth)acrylate. In certain preferred embodiments, the non-ionic ethylenically unsaturated monomers are selected from methyl methacrylate and butyl methacrylate. In certain embodiments, the core comprises polymerized units of non-ionic ethylenically unsaturated monomers in an amount of from 40 to 80 weight %, preferably from 50 to 70 weight %, and more preferably from 55 to 65 weight %, based on the total weight of the core polymer.

The voided latex particles suitable for use in the present invention also include at least one shell polymer. The at least one shell polymer(s) comprise polymerized units derived from non-ionic ethylenically unsaturated monomers and polyethylenically unsaturated monomers. In certain embodiments, at least one shell polymer optionally comprises polymerized units derived from at least one of monoethylenically unsaturated monomers containing at least one carboxylic acid group and monoethylenically unsaturated monomers containing at least one “non-carboxylic” acid group. In certain embodiments, the shell portion of the voided latex particles are polymerized in a single stage, preferably in two stages, and more preferably in at least three stages. As used herein, the term “outermost shell” refers to the composition of the final distinct polymerization stage used to prepare the voided latex particles. In certain embodiments wherein the outermost shell is provided by a multistage polymerization process, the outermost shell comprises at least 25 weight %, preferably at least 35 weight %, and more preferably at least 45 weight % of the total shell portion of the voided latex particle.

Suitable non-ionic ethylenically unsaturated monomers for the shell polymer(s) include, for example, vinyl acetate, acrylonitrile, methacrylonitrile, nitrogen containing ring compound unsaturated monomers, vinylaromatic monomers, ethylenic monomers and selected (meth)acrylic acid derivatives. Suitable (meth)acrylic acid derivatives include, for example, (C₁-C₂₂)alkyl (meth)acrylate, substituted (meth)acrylate, and substituted (meth)acrylamide monomers. In certain preferred embodiments, the (meth)acrylic acid derivatives are selected from methyl acrylate, methyl methacrylate, ethyl acrylate, ethyl methacrylate, butyl acrylate, butyl methacrylate, isobutyl acrylate, isobutyl methacrylate, hydroxyethyl methacrylate, hydroxypropyl methacrylate, dimethylaminoethyl methacrylate, dimethylaminopropyl methacrylamide, and mixtures thereof. Suitable vinylaromatic monomers include, for example, styrene, α-methylstyrene, vinyltoluene, alkyl-substititued styrene (such as t-butylstyrene and ethylvinylbenzene), and halogenated styrenes (such as chlorostyrene and 3,5-bis (trifuoromethyl)styrene). In certain preferred embodiments, the vinylaromatic monomers are selected from styrene, ethylvinylbenzene, t-butylstrene, and mixtures thereof. In certain embodiments, the shell polymer(s) comprise polymerized units of non-ionic ethylenically unsaturated monomers in an amount of from 55 to 85 weight %, preferably from 60 to 80 weight %, and more preferably from 65 to 75 weight %, based on the total weight of the shell polymer(s).

Suitable polyethylenically unsaturated monomers for the shell polymer(s) include, for example, di(meth)acrylates, tri(meth)acrylates, tetra(meth)acrylates, polyallylic monomers, polyvinylic monomers, and (meth)acrylic monomers having mixed ethylenic functionality. Suitable polyvinylic monomers include, for example, diethyleneglycol divinyl ether, divinylbenzene, divinyl ketone, divinylpyridine, divinyl sulfide, divinyl sulfone, divinyltoluene, divinylxylene, glycerol trivinyl ether, trivinylbenZene, 1,2,4-trivinylcyclohexane, N,N′-ethylenebisacrylamide, partially fluorinated α,Ω-dienes (such as CF₂═CFCF₂CF₂CH₂CH═CH₂), trifluoroalkadienes, trifluorodivinylbenzenes, and fluorinated divinyl ethers of fluorinated 1,2-ethanediol. In certain preferred embodiments, the polyvinylic monomer comprises divinylbenzene. Suitable (meth)acrylic monomers having mixed ethylenic functionality include, for example, the acrylate ester of neopentyl glycol monodicyclopentenyl ether, allyl acryloxypropionate, allyl acrylate, allyl methacrylate, crotyl acrylate, crotyl methacrylate, 3-cyclohexenylmethyleneoxyethyl acrylate, 3-cyclohexenylmethyleneoxyethyl methacrylate, dicyclopentadienyloxyethyl acrylate, dicyclopentadienyloxyethyl methacrylate, dicyclopentenyl acrylate, dicyclopentenyl methacrylate, dicyclopentenyloxyethyl acrylate, dicycol pentenyloxyethyl methacrylate, methacrylate ester of neopentyl glycol monodicyclopentenyl ether, methallyl acrylate, trimethylolpropane diallyl ether mono-acrylate, trimethylolpropane diallyl ether mono-methacrylate, and N-allyl acrylamide. In certain preferred embodiments, the (meth)acrylic monomers having mixed ethylenic functionality comprise allyl methacrylate. In certain embodiments, the shell polymer(s) comprise polymerized units of polyethylenically unsaturated monomers in an amount of from 15 to 45 weight %, preferably from 20 to 35 weight %, and more preferably from 22 to 30 weight %, based on the total weight of the shell polymer(s). In certain embodiments, the outermost shell comprises polymerized units of polyethylenically unsaturated monomers in an amount of from 10 to 100 weight %, preferably from 15 to 70 weight %, and more preferably from 20 to 60 weight %, based on the weight of the outermost shell polymer.

Suitable monoethylenically unsaturated monomers containing at least one carboxylic acid group for the shell polymer(s) include, for example, (meth)acrylic acid, (meth)acryloxypropionic acid, itaconic acid, aconitic acid, maleic acid, fumaric acid, crotonic acid, citraconic acid, maleic anhydride monomethyl maleate, monomethyl fumarate, and monomethyl itaconate, and other derivatives such as corresponding anhydride, amides, and esters. In certain preferred embodiments, the monoethylenically unsaturated monomers containing at least one carboxylic acid group are selected from acrylic acid and methacrylic acid. In certain embodiments, the shell polymer(s) comprises polymerized units of monoethylenically unsaturated monomers containing at least one carboxylic acid group in an amount of from 0.1 to 10 weight %, preferably from 0.3 to 7.5 weight %, and more preferably from 0.5 to 5 weight %, based on the total weight of the shell polymer(s).

Suitable monoethylenically unsaturated monomers containing at least one “non-carboxylic” acid group for the shell polymer(s) include, for example, allylsulfonic acid, allylphosphonic acid, allyloxybenzenesulfonic acid, 2-acrylamido-2-methylpropanesulfonic acid (the acryonym “AMPS” for this monomer is a trademark of LubriZol Corporation, Wickliffe, Ohio, USA), 2-hydroxy-3-(2-propenyloxy)propanesulfonic acid, 2-methyl-2-propene-1-sulfonic acid, 2-methacrylamido-2-methyl-1-propanesulfonic acid, 3-methacrylamido-2-hydroxy-1-propanesulfonic acid, 3-sulfopropyl acrylate, 3-sulfopropyl methacrylate, isopropenylphosphonic acid, vinylphosphonic acid, phosphoethyl methacrylate, styrenesulfonic acid, vinylsulfonic, acid and the alkali metal and ammonium salts thereof. In certain preferred embodiments, the monoethylenically unsaturated monomers containing at least one “non-carboxylic” acid group are selected from 2-acrylamido-2-methylpropanesulfonic acid, styrenesulfonic acid, and sodium styrene sulfonate. In certain embodiments, the shell polymer(s) comprise polymerized units of monoethylenically unsaturated monomers containing at least one “non-carboxylic” acid group in an amount of from 0.1 to 10 weight %, preferably from 0.5 to 7.5 weight %, and more preferably from 1 to 5 weight %, based on the total weight of the shell polymer(s).

The shell polymer(s) of the latex particles suitable for use in the present invention have T_{g} values which are high enough to support to support the void within the latex particle. In certain embodiments, the T_{g} values of at least one shell are greater than 50° C., preferably greater than 60° C., and more preferably greater than 70° C.

In certain embodiments, the core polymer and shell polymer are made in a single polymerization step. In certain other embodiments, the core polymer and shell polymer are made in a sequence of polymerization steps. Suitable polymerization techniques for preparing the voided latex particles contained in the inventive personal care compositions include, for example, sequential emulsion polymerization. In certain embodiments, the monomers used in the emulsion polymerization of the shell polymer of the voided latex particles comprise one or more non-ionic ethylenically unsaturated monomer. Aqueous emulsion polymerization processes typically are conducted in an aqueous reaction mixture, which contains at least one monomer and various synthesis adjuvants, such as the free radical sources, buffers, and reductants in an aqueous reaction medium. In certain embodiments, a chain transfer agent may be used to limit molecular weight. The aqueous reaction medium is the continuous fluid phase of the aqueous reaction mixture and contains more than 50 weight % water and optionally one or more water miscible solvents, based on the weight of the aqueous reaction medium. Suitable water miscible solvents include, for example, methanol, ethanol, propanol, acetone, ethylene glycol ethyl ethers, propylene glycol propyl ethers, and diacetone alcohol.

In certain embodiments, the void of the latex particles is prepared by swelling the core with a swelling agent containing one or more volatile components. The swelling agent permeates the shell to swell the core. The volatile components of the swelling agent can then be removed by drying the latex particles, causing a void to be formed within the latex particles. In certain embodiments, the swelling agent is an aqueous base. Suitable aqueous bases useful for swelling the core include, for example, ammonia, ammonium hydroxide, alkali metal hydroxides, such as sodium hydroxide, or a volatile amine such as trimethylamine or triethylamine. In certain embodiments, the voided latex particles are added to the composition with the swelling agent present in the core. When the latex particles are added to the composition with the swelling agent present in the core, the volatile components of the swelling agent will be removed upon drying of the composition. In certain other embodiments, the voided latex particles are added to the composition after removing the volatile components of the swelling agent.

In certain embodiments, the voided latex particles contain a void with a void fraction of from 1% to 70%, preferably from 5% to 50%, more preferably from 10% to 40%, and even more preferably from 25% to 35%. The void fractions are determined by comparing the volume occupied by the latex particles after they have been compacted from a dilute dispersion in a centrifuge to the volume of non-voided particles of the same composition. In certain embodiments, the voided latex particles have a particle size of from 400 nm to 1500 nm, as measured by a Brookhaven BI-90. In certain embodiments, the voided latex particles have a particle size of from 400 nm to 800 nm, preferably from 400 nm to 700 nm, more preferably from 400 nm to 600 nm, and even more preferably from 400 nm to 550 nm, as measured by a Brookhaven BI-90. In certain embodiments, the voided latex particles have a particle size of from 800 nm to 1500 nm, preferably from 400 nm to 700 nm, more preferably from 400 nm to 600 nm, and even more preferably from 400 nm to 550 nm, as measured by a Brookhaven BI-90

A person of ordinary skill in the art can readily determine the effective amount of the voided latex particles that should be used in a particular composition in order to provide the benefits described herein (e.g., improved light scattering to compositions containing inorganic metal oxide particles, and providing a long lasting whitening effect when applied to skin), via a combination of general knowledge of the applicable field as well as routine experimentation where needed. By way of non-limiting example, the amount of voided latex particles in the composition of the invention may be in the range of from 0.5 to 20 solids weight %, preferably from 1 to 7 solids weight %, more preferably from 1 to 2 solids weight %, based on the total weight of the composition.

The personal care compositions of the present invention also comprise inorganic metal oxide particles. Suitable inorganic metal oxide particles include, for example, zinc oxide (ZnO), titanium dioxide (TiO₂), and mixtures thereof. In certain embodiments, the inorganic metal oxide particles are pigment grade ZnO or pigment grade TiO₂ that produce a white appearance caused by light scattering. In certain embodiments, pigment grade inorganic metal oxide particles having good pigmentation properties and have a particle size in the range of from 250 nm to 1000 nm, preferably from 250 nm to 500 nm, and more preferably from 250 nm to 350 nm. Suitable ZnO and TiO₂ particles include, for example, those commercially available under the trade names AQUASPERSABIL Rutile TiO₂ and OLEOSPERSE TiO₂ from Presperse Corporation, and TITANIX TiO₂ from Tyca Corporation. In certain embodiments, the skin care compositions include inorganic metal oxide particles in an amount of from 0.1 to 20 weight %, preferably 0.1 to 10 weight %, and more preferably 0.1 to 5 weight %, based on the total weight of the composition.

Compositions of the invention also include a dermatologically acceptable carrier. Such material is typically characterized as a carrier or a diluent that does not cause significant irritation to the skin and does not negate the activity and properties of active agent(s) in the composition. Examples of dermatologically acceptable carriers that are useful in the invention include, without limitation, water, such as deionized or distilled water, emulsions, such as oil-in-water or water-in-oil emulsions, alcohols, such as ethanol, isopropanol or the like, glycols, such as propylene glycol, glycerin or the like, creams, aqueous solutions, oils, ointments, pastes, gels, lotions, milks, foams, suspensions, powders, or mixtures thereof. In some embodiments, the composition contains from about 99.99 to about 50 percent by weight of the dermatologically acceptable carrier, based on the total weight of the composition.

The personal care composition of the invention may also include, for instance, a thickener, additional emollients, an emulsifier, a humectant, a surfactant, a suspending agent, a film forming agent, a lower monoalcoholic polyol, a high boiling point solvent, a propellant, a mineral oil, silicon feel modifiers, or mixtures thereof. The amount of optional ingredients effective for achieving the desired property provided by such ingredients can be readily determined by one skilled in the art.

Other additives may be included in the compositions of the invention such as, but not limited to, abrasives, absorbents, aesthetic components such as fragrances, pigments, colorings/colorants, essential oils, skin sensates, astringents (e.g., clove oil, menthol, camphor, eucalyptus oil, eugenol, menthyl lactate, witch hazel distillate), preservatives, anti-caking agents, a foam building agent, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, and vitamins (e.g., Vitamin C) and derivatives thereof. The amount of option ingredients effective for achieving the desired property provided by such ingredients can be readily determined by one skilled in the art.

As noted above, personal care compositions of the present invention are highly effective as skin lightening agents. They exhibit skin lightening attributes on par with, if not better than previously known compositions for personal care applications, without the disadvantage of a short timeframe of effectiveness after application. Thus, in one aspect the present invention provides that the personal care compositions may be used in a method for lightening skin tone. In another aspect, the present invention provides a method for improving the visible light scattering of a composition by adding the voided latex particles to a composition comprising at least one pigment grade inorganic metal oxide particle. In practicing the methods of the invention, the personal care compositions are generally administered topically by applying or spreading the compositions onto the skin. A person of ordinary skill in the art can readily determine the frequency with which the compositions should be applied. The frequency may depend, for example, on the level of skin lightening that an individual is likely to desire. By way of non-limiting example, administration on a frequency of at least once per day may be desirable.

Some embodiments of the invention will now be described in detail in the following Examples.

### EXAMPLES

### Example 1

### Preparation of Exemplary and Comparative Copolymer Particles

Exemplary voided latex particles in accordance with the present invention and comparative particles in the Examples below contain the components recited in Table 1.

TABLE 1
Exemplary and Comparative Copolymer Particles
Monomer (wt %)

| | |
|---|---|
| Core (4.7%): | 60 MMA/40 MAA |
| Shell 1 (22.1%): | 8.5 BMA/88.5 MMA/3 MAA |
| Shell 2 (26.8%): | 94.9 Sty/5.1 DVB |
| Shell 3 (46.4%): | 46.2 Sty/51.1 DVB/2.7 SSS |

MMA = methyl methacrylate
BMA = butyl methacrylate
MAA = methacrylic acid
Sty = styrene
DVB = divinylbenzene
SSS = sodium styrene sulfonate

To a 3-liter, 4-neck round bottom flask equipped with overhead stirrer, thermocouple, heating mantle, adapter inlet, Claisen head fitted with a water condenser and nitrogen inlet, and an inlet adapter, was added 875.3 grams (g) deionized water which was heated to 84° C. under nitrogen. To the heated water was added 0.30 g acetic acid, 1.70 g sodium persulfate in 15.5 g of deionized water followed by the addition of an aqueous dispersion of 31% poly(MMA/MAA//60/40) acrylic seed (core) polymer, having an average particle diameter of approximately 110 to 220 nm. To this heated mixture at 82° C., a monomer emulsion containing 71.5 g deionized water, 2.1 g aqueous solution of 23% SDBS, 91.6 g MMA, 8.9 g BMA and 3.1 g MAA was metered in over 90 minutes followed by a deionized water rinse. Next, a solution of 0.65 g sodium persulfate in 32.8 g deionized water was added over 90 minutes and the reaction temperature was raised to 90° C. concurrent with the addition of a second monomer emulsion containing 48.3 g deionized water, 0.35 g aqueous solution of 23%SDBS, 120.5 g Sty, 6.45 g DVB and 0.70 g linseed oil fatty acid over 30 minutes. At the completion of addition of the second monomer emulsion, 8.0 g aqueous 28% ammonium hydroxide was added, and hold for 10 min. To the reaction mixture at 91° C. was added, over 60 minutes, a third monomer emulsion containing 100.5 g deionized water, 1.0 g aqueous solution of 23%SDBS, 104.2 g Sty and 115.25 g DVB, followed by a deionized water rinse. The reactor contents were held at 91° C. for 30 minutes, then 5.8 g of aqueous solution containing 0.10 g of FeSO4.7H20 and 0.10 g of versene was added followed by the concurrent addition over 60 minutes of 5.10 g of t-butylhydrogen peroxide (70%) in 19.0 g of deionized water and 2.6 g isoascorbic acid in 19.0 g deionized water, to the reactor maintained at 91° C. The latex was cooled to room temperature and then filtered.

### Example 2

### Characterization of Exemplary and Comparative Latex Particles

Voided latex particles as prepared in Example 1 were evaluated for particle size and percent void fraction, as shown in Table 2.

TABLE 2
Characterization of Latex Particles

| | Particle Size | % Void |
|---|---|---|
| Sample | (nm) | Fraction |
| Polymer A | 400 | 30 |
| Polymer B | 550 | 34 |
| Polymer C | 1030 | 26 |
| Polymer D* | 350 | 24 |

*Comparative

The particle size was measured using a Brookhaven BI-90. The percent void fraction of the latex particles was measured by making a 10% by weight dispersion of each sample with propylene glycol, which was then mixed and poured into a weight-per-gallon cup which was capped and weighed. A 10% water blank was also measured, and the difference in the weight was used to calculate the density of the sample, from which the percent void fraction was determined.

### Example 3

### Preparation of Exemplary Skin Lightening Formulations

Exemplary skin lightening formulations according to the present invention contain the components recited in Table 3.

TABLE 3
Exemplary Skin Lightening Formulations

| | | | | | | | | | |
|---|---|---|---|---|---|---|---|---|---|
| | | E1 | E2 | E3 | E4 | E5 | E6 | E7 | E8 |
| Trade Name | INCI Name | w/w % | w/w % | w/w % | w/w % | w/w % | w/w % | w/w % | w/w % |

Phase I

| | | | | | | | | | |
|---|---|---|---|---|---|---|---|---|---|
| Water | — | q.s. to | q.s. to | q.s. to | q.s. to | q.s. to | q.s. to | q.s. to | q.s. to |
| | | 100 | 100 | 100 | 100 | 100 | 100 | 100 | 100 |
| Carbopol | Carbomer | 0.15 | 0.15 | 0.15 | 0.15 | 0.15 | 0.15 | 0.15 | 0.15 |
| Ultrez 10¹ | | | | | | | | | |
| 1,3- | Butylene | 2.00 | 2.00 | 2.00 | 2.00 | 2.00 | 2.00 | 2.00 | 2.00 |
| Butanediol | Glycol | | | | | | | | |
| Aquaspersabil | Titanum | 1.00 | 1.00 | 2.00 | 2.00 | 1.00 | 2.00 | 1.00 | 2.00 |
| Rutile TiO₂² | Dioxide | | | | | | | | |
| Polymer A | — | 1.00 | 2.00 | 1.00 | 2.00 | — | — | — | — |
| | | as | as | as | as | | | | |
| | | solids | solids | solids | solids | | | | |
| Polymer B | — | — | — | — | — | 1.00 | 2.00 | — | — |
| | | | | | | as | as | | |
| | | | | | | solids | solids | | |
| Polymer C | — | — | — | — | — | — | — | 1.00 | 2.00 |
| | | | | | | | | as | as |
| | | | | | | | | solids | solids |

Phase II

| | | | | | | | | | |
|---|---|---|---|---|---|---|---|---|---|
| Procol CS- | Cetearyl | 2.00 | 2.00 | 2.00 | 2.00 | 2.00 | 2.00 | 2.00 | 2.00 |
| 20D³ | Alcohol (and) | | | | | | | | |
| | Ceteareth 20 | | | | | | | | |
| Glyceryl | Glyceryl | 1.00 | 1.00 | 1.00 | 1.00 | 1.00 | 1.00 | 1.00 | 1.00 |
| Stearate | Stearate | | | | | | | | |
| Isopropyl | Isopropyl | 3.00 | 3.00 | 3.00 | 3.00 | 3.00 | 3.00 | 3.00 | 3.00 |
| Myristate | Myristate | | | | | | | | |
| Ritamollient | Caprylic/ | 3.00 | 3.00 | 3.00 | 3.00 | 3.00 | 3.00 | 3.00 | 3.00 |
| CCT⁴ | Capric | | | | | | | | |
| | Triglyceride | | | | | | | | |

Phase III

| | | | | | | | | | |
|---|---|---|---|---|---|---|---|---|---|
| Optiphen⁵ | Phenoxyethanol | 1.00 | 0.00 | 1.00 | 1.00 | 1.00 | 1.00 | 1.00 | 1.00 |
| | (and) Caprylyl | | | | | | | | |
| | glycol | | | | | | | | |

¹Available from Lubrizol
²Available from Presperse
³Available from Protameen
⁴Available from RITA
⁵Available from Ashland

The skin lightening formulations were prepared by adding Ultrez 10 and 1,3-butanediol to water and mixing until the Ultrez 10 was completely dissolved. The remaining components of Phase I were then added to the mixture. Phase II components were mixed separately and heated to 70° C. to ensure that all components were melted. Phases I and II were then combined while mixing and cooled to about 50° C., at which point Phase III components were added to the mixture. The mixture was allowed to cool to about 30° C., and the pH was adjusted to a pH of about 5.5-6.0 by adding triethanolamine dropwise.

### Example 4

### Preparation of Comparative Skin Lightening Formulations

Comparative skin lightening formulations contain the components recited in Table 4.

TABLE 4
Comparative Skin Lightening Formulations

| | | | | | | | | |
|---|---|---|---|---|---|---|---|---|
| | | C1 | C2 | C3 | C4 | C5 | C6 | C7 |
| Trade Name | INCI Name | w/w % | w/w % | w/w % | w/w % | w/w % | w/w % | w/w % |

Phase I

| | | | | | | | | |
|---|---|---|---|---|---|---|---|---|
| Water | — | q.s. to | q.s. to | q.s. to | q.s. to | q.s. to | q.s. to | q.s. to |
| | | 100 | 100 | 100 | 100 | 100 | 100 | 100 |
| Carbopol | Carbomer | 0.15 | 0.15 | 0.15 | 0.15 | 0.15 | 0.15 | 0.15 |
| Ultrez 10¹ | | | | | | | | |
| 1,3- | Butylene | 2.00 | 2.00 | 2.00 | 2.00 | 2.00 | 2.00 | 2.00 |
| Butanediol | Glycol | | | | | | | |
| Aquaspersabil | Titanum | — | 1.00 | 2.00 | — | 1.00 | 2.00 | 4.00 |
| Rutile TiO₂² | Dioxide | | | | | | | |
| Polymer A | — | — | — | — | 2.00 | — | — | — |
| | | | | | as | | | |
| | | | | | solids | | | |
| Polymer D | — | — | — | — | — | 1.00 | 2.00 | — |
| | | | | | | as | as | |
| | | | | | | solids | solids | |

Phase II

| | | | | | | | | |
|---|---|---|---|---|---|---|---|---|
| Procol CS- | Cetearyl | 2.00 | 2.00 | 2.00 | 2.00 | 2.00 | 2.00 | 2.00 |
| 20D³ | Alcohol (and) | | | | | | | |
| | Ceteareth 20 | | | | | | | |
| Glyceryl | Glyceryl | 1.00 | 1.00 | 1.00 | 1.00 | 1.00 | 1.00 | 1.00 |
| Stearate | Stearate | | | | | | | |
| Isopropyl | Isopropyl | 3.00 | 3.00 | 3.00 | 3.00 | 3.00 | 3.00 | 3.00 |
| Myristate | Myristate | | | | | | | |
| Ritamollient | Caprylic/ | 3.00 | 3.00 | 3.00 | 3.00 | 3.00 | 3.00 | 3.00 |
| CCT⁴ | Capric | | | | | | | |
| | Triglyceride | | | | | | | |

Phase III

| | | | | | | | | |
|---|---|---|---|---|---|---|---|---|
| Optiphen⁵ | Phenoxyethanol | 1.00 | 0.00 | 1.00 | 1.00 | 1.00 | 1.00 | 1.00 |
| | (and) Caprylyl | | | | | | | |
| | glycol | | | | | | | |

¹Available from Lubrizol
²Available from Presperse
³Available from Protameen
⁴Available from RITA
⁵Available from Ashland

The skin lightening formulations were prepared by adding Ultrez 10 and 1,3-butanediol to water and mixing until the Ultrez 10 was completely dissolved. The remaining components of Phase I were then added to the mixture. Phase II components were mixed separately and heated to 70° C. to ensure that all components were melted. Phases I and II were then combined while mixing and cooled to about 50° C., at which point Phase III components were added to the mixture. The mixture was allowed to cool to about 30° C., and the pH was adjusted to a pH of about 5.5-6.0 by adding triethanolamine dropwise.

### Example 5

### Whitening Study of Skin Lightening Formulations Including 1 Weight % TiO₂ and Exemplary 400 nm Particles

The whitening effect of exemplary formulations including 1 weight % TiO₂ and Polymer A particles, and comparative formulations including 1 weight % TiO₂, as prepared in Examples 3 and 4 were measuring using an in vitro technique substantially in accordance with the following protocol. The in vitro whitening test was conducted in an environment controlled room with temperature at 70-75° F. and relative humidity at 48-55%. The substrate used for the test was a Leneta Form 2A Opacity Card, and reflectance was measured using Novo-Shade Duo 45/0 reflectometer. A BYK 2″ wide 3 mil Wet Film Bird Applicator was used to make the drawdown film.

The opacity card was placed on a vacuum plate with vacuum on. For each testing formulation, a drawdown film was made using the 3 mil wet film bird applicator. Reflectance on both black and white side of the card was measured in Opacity mode using the reflectometer with a proper guard to avoid the drawdown film being touched. The first reflectance measurement was made at 1 minute after the drawdown film was made on both black and white sides of the card, followed by measurement at 15 minutes intervals up to 120 minutes. Two additional measurements were made at 4 hours and 24 hours after the drawdown film was made.

Reflectance data on both black and white chart was collected and the contrast ratio was calculated; only reflectance on the black chart is adopted for the analysis due to correlation between contrast ratio and reflectance number.

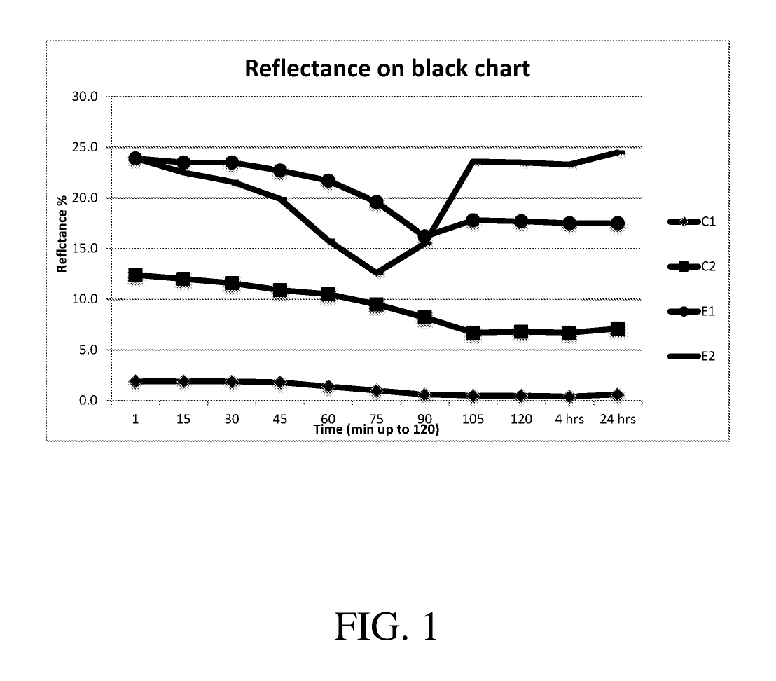

The results of the reflectance measurements for films including 1% TiO₂ are shown in Table 5 and FIG. 1 as percent reflectance.

TABLE 5
Reflectance of Exemplary and Comparative Films Including
1 Weight % TiO₂ and Exemplary 400 nm Particles

| Time | C1 | C2 | E1 | E2 |
|---|---|---|---|---|
| (min up to 120) | (% ref) | (% ref) | (% ref) | (% ref) |
| 1 | 1.9 | 12.4 | 23.9 | 23.9 |
| 15 | 1.9 | 12.0 | 23.5 | 22.5 |
| 30 | 1.9 | 11.6 | 23.5 | 21.6 |
| 45 | 1.8 | 10.9 | 22.7 | 19.9 |
| 60 | 1.4 | 10.5 | 21.7 | 15.8 |
| 75 | 1.0 | 9.5 | 19.6 | 12.6 |
| 90 | 0.6 | 8.2 | 16.2 | 15.5 |
| 105 | 0.5 | 6.7 | 17.8 | 23.6 |
| 120 | 0.5 | 6.8 | 17.7 | 23.5 |
| 4 hrs | 0.4 | 6.7 | 17.5 | 23.3 |
| 24 hrs | 0.6 | 7.1 | 17.5 | 24.5 |

The results demonstrate that, when used alone, 1% TiO₂ delivers initial whitening performance, but fades upon drying, presumably due to TiO₂ agglomeration, and that the exemplary whitening formulations prepared in accordance with the present invention provide a constant and prolonged reflectance value significantly higher than comparative formulations.

### Example 6

### Whitening Study of Skin Lightening Formulations Including 2 Weight % TiO₂ and Exemplary 400 nm Particles

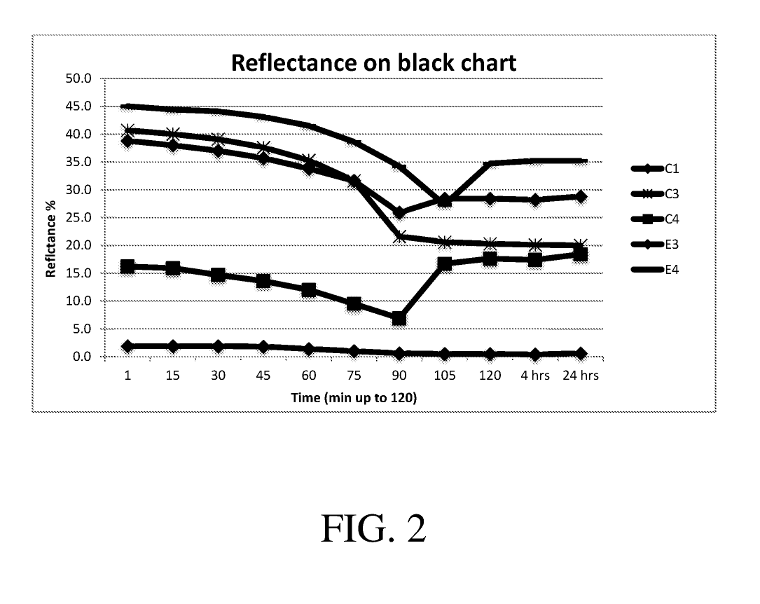

The whitening effect of exemplary formulations including 2 weight % TiO₂ and Polymer A particles, and comparative formulations including 2 weight % TiO₂, as prepared in Examples 3 and 4 were measuring using an in vitro technique substantially according to the protocol described in Example 5. The results of the reflectance measurements are shown in Table 6 and FIG. 2 as percent reflectance.

TABLE 6
Reflectance of Exemplary and Comparative Films Including
2 Weight % TiO₂ and Exemplary 400 nm Particles

| Time | C1 | C3 | C4 | E3 | E4 |
|---|---|---|---|---|---|
| (min up to 120) | (% ref) | (% ref) | (% ref) | (% ref) | (% ref) |
| 1 | 1.9 | 40.7 | 16.2 | 38.8 | 45.0 |
| 15 | 1.9 | 40.0 | 15.9 | 38.0 | 44.4 |
| 30 | 1.9 | 39.1 | 14.7 | 37.0 | 44.1 |
| 45 | 1.8 | 37.6 | 13.6 | 35.7 | 43.1 |
| 60 | 1.4 | 35.3 | 12.0 | 33.8 | 41.5 |
| 75 | 1.0 | 31.6 | 9.5 | 31.6 | 38.6 |
| 90 | 0.6 | 21.6 | 6.9 | 25.9 | 34.2 |
| 105 | 0.5 | 20.6 | 16.7 | 28.4 | 27.3 |
| 120 | 0.5 | 20.3 | 17.6 | 28.4 | 34.7 |
| 4 hrs | 0.4 | 20.1 | 17.4 | 28.2 | 35.2 |
| 24 hrs | 0.6 | 20.0 | 18.4 | 28.8 | 35.2 |

The results demonstrate that, when used alone, 2% TiO₂ delivers initial whitening performance, but fades upon drying, presumably due to TiO₂ agglomeration, and that the exemplary whitening formulations prepared in accordance with the present invention provide a constant and prolonged reflectance value significantly higher than comparative formulations.

### Example 7

### Whitening Study of Skin Lightening Formulations Including 1 Weight % TiO₂, and Exemplary 400 nm Particles or Exemplary 550 nm Particles

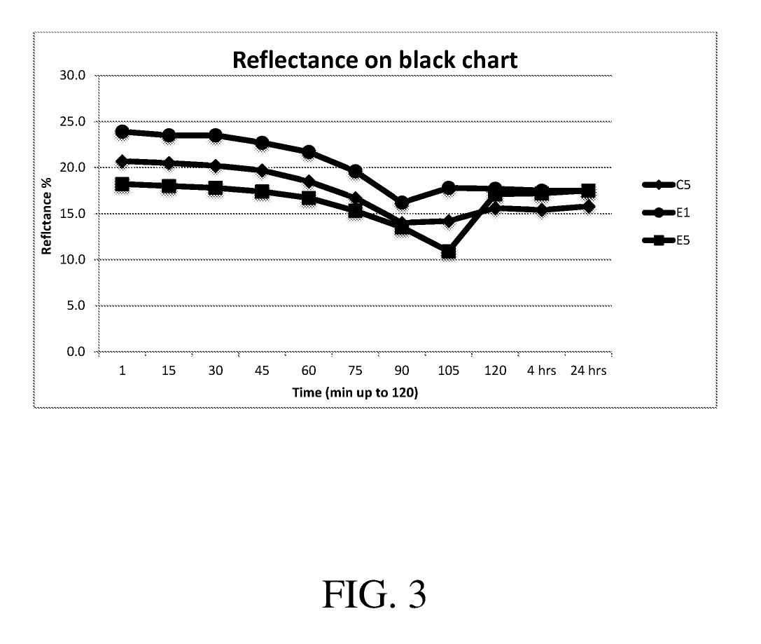

The whitening effect of exemplary formulations including 1 weight % TiO₂ and Polymer A particles (E1), exemplary formulations including 1 weight % TiO₂ and Polymer B particles (E5), and comparative formulations including 1 weight % TiO₂ and Polymer C particles (C5), as prepared in Examples 3 and 4 were measuring using an in vitro technique substantially according to the protocol described in Example 5. The results of the reflectance measurements are shown in Table 7 and FIG. 3 as percent reflectance.

TABLE 7
Reflectance of Exemplary and Comparative Films
Including 1 Weight % TiO₂, and Exemplary 400
nm Particles or Exemplary 550 nm Particles

| Time | C5 | E1 | E5 |
|---|---|---|---|
| (min up to 120) | (% ref) | (% ref) | (% ref) |
| 1 | 20.7 | 23.9 | 18.2 |
| 15 | 20.5 | 23.5 | 18.0 |
| 30 | 20.2 | 23.5 | 17.8 |
| 45 | 19.7 | 22.7 | 17.4 |
| 60 | 18.5 | 21.7 | 16.7 |
| 75 | 16.7 | 19.6 | 15.3 |
| 90 | 14.0 | 16.2 | 13.5 |
| 105 | 14.2 | 17.8 | 10.9 |
| 120 | 15.6 | 17.7 | 17.1 |
| 4 hrs | 15.4 | 17.5 | 17.2 |
| 24 hrs | 15.8 | 17.5 | 17.5 |

The results demonstrate that whitening formulations including 1 weight % TiO₂ prepared in accordance with the present invention including particles of 400 nm and 550 nm demonstrate significantly higher reflectance values as compared with comparative formulations including particles of 350 nm.

### Example 8

### Whitening Study of Skin Lightening Formulations Including 2 Weight % TiO₂, and Exemplary 400 nm Particles or Exemplary 550 nm Particles

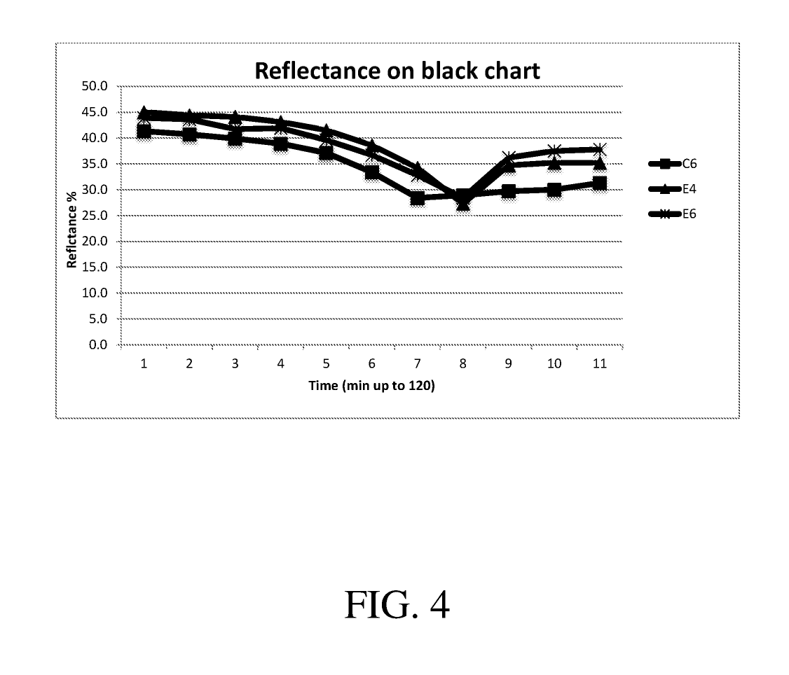

The whitening effect of exemplary formulations including 2 weight % TiO₂ and Polymer A particles (E4), exemplary formulations including 2 weight % TiO₂ and Polymer B particles (E6), and comparative formulations including 2 weight % TiO₂ and Polymer C particles (C6), as prepared in Examples 3 and 4 were measuring using an in vitro technique substantially according to the protocol described in Example 5. The results of the reflectance measurements are shown in Table 8 and FIG. 4 as percent reflectance.

TABLE 8
Reflectance of Exemplary and Comparative Films
Including 2 Weight % TiO₂, and Exemplary 400
nm Particles or Exemplary 550 nm Particles

| Time | C6 | E4 | E6 |
|---|---|---|---|
| (min up to 120) | (% ref) | (% ref) | (% ref) |
| 1 | 41.3 | 45.0 | 43.9 |
| 15 | 40.7 | 44.4 | 43.6 |
| 30 | 39.9 | 44.1 | 41.7 |
| 45 | 38.9 | 43.1 | 41.9 |
| 60 | 37.1 | 41.5 | 39.6 |
| 75 | 33.4 | 38.6 | 36.7 |
| 90 | 28.4 | 34.2 | 32.8 |
| 105 | 28.9 | 27.3 | 28.4 |
| 120 | 29.7 | 34.7 | 36.2 |
| 4 hrs | 30.0 | 35.2 | 37.5 |
| 24 hrs | 31.3 | 35.2 | 37.8 |

The results demonstrate that whitening formulations including 1 weight % TiO₂ prepared in accordance with the present invention including particles of 400 nm and 550 nm demonstrate significantly higher reflectance values as compared with comparative formulations including particles of 350 nm.

### Example 9

### Whitening Study of Exemplary Skin Lightening Formulations Including 1 Weight % Exemplary 1000 nm Particles and TiO₂

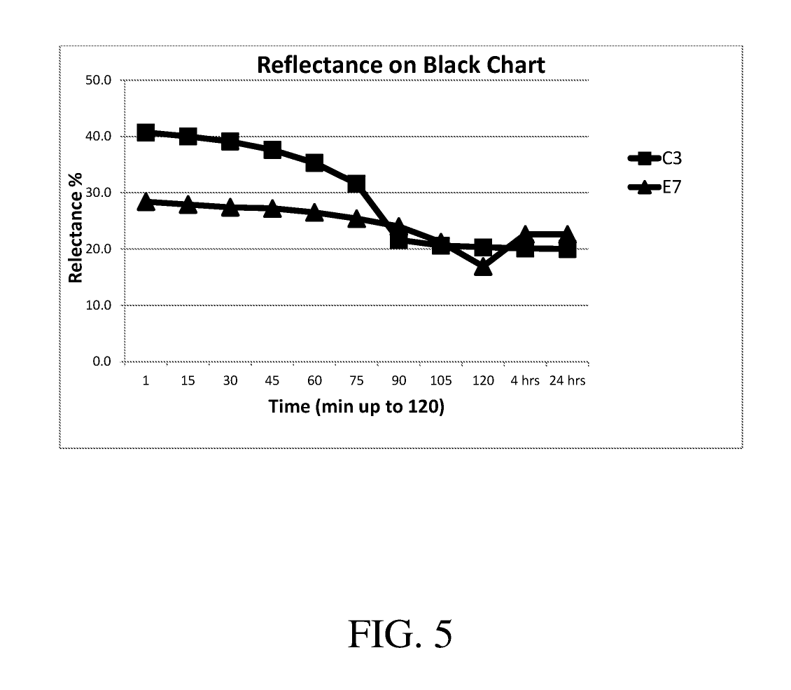

The whitening effect of exemplary formulations including 1 weight % TiO₂ and 1 weight % Polymer C particles (E7) and comparative formulations including 2 weight % TiO₂ (C3) as prepared in Examples 3 and 4 were measuring using an in vitro technique substantially according to the protocol described in Example 5. The results of the reflectance measurements are shown in Table 9 and FIG. 5 as percent reflectance.

TABLE 9
Reflectance of Exemplary Films Including 1 Weight
% TiO₂ and 1 Weight % Exemplary 1000 nm Particles,
and Comparative Films Including 2 Weight % TiO₂

| Time | C3 | E7 |
|---|---|---|
| (min up to 120) | (% ref) | (% ref) |
| 1 | 40.7 | 28.4 |
| 15 | 40.0 | 27.9 |
| 30 | 39.1 | 27.4 |
| 45 | 37.6 | 27.2 |
| 60 | 35.3 | 26.5 |
| 75 | 31.6 | 25.4 |
| 90 | 21.6 | 24.0 |
| 105 | 20.6 | 21.2 |
| 120 | 20.3 | 16.9 |
| 4 hrs | 20.1 | 22.6 |
| 24 hrs | 20.0 | 22.6 |

The results demonstrate that, when used alone, 2% TiO₂ delivers initial whitening performance, but fades upon drying, presumably due to TiO₂ agglomeration, and that the exemplary whitening formulations prepared in accordance with the present invention provide a constant and prolonged reflectance value significantly higher than comparative formulations.

### Example 10

### Whitening Study of Exemplary Skin Lightening Formulations Including 1 Weight % Exemplary 1000 nm Particles and TiO₂

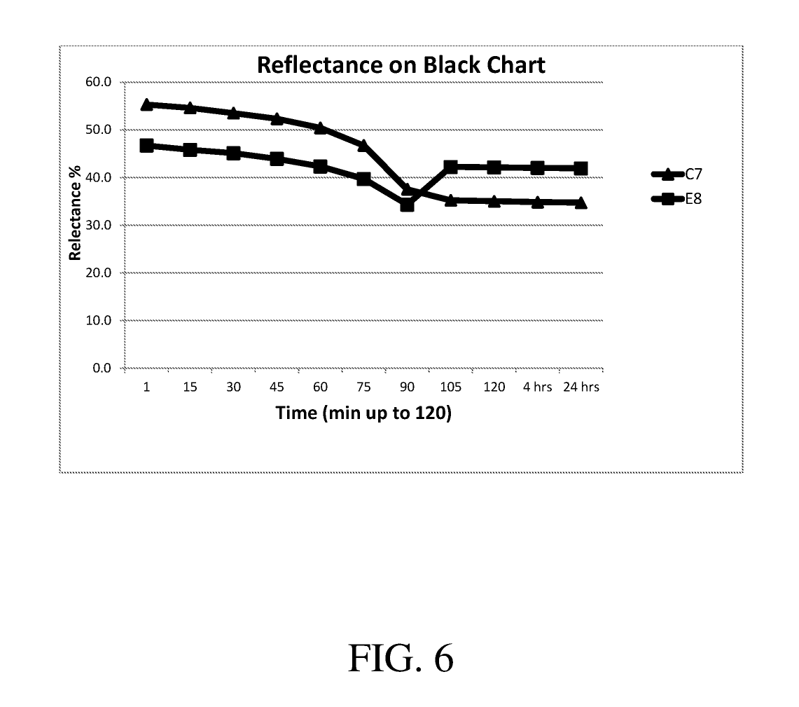

The whitening effect of exemplary formulations including 2 weight % TiO₂ and 2 weight % Polymer C particles (E8) and comparative formulations including 4 weight % TiO₂ (C7) as prepared in Examples 3 and 4 were measuring using an in vitro technique substantially according to the protocol described in Example 5. The results of the reflectance measurements are shown in Table 10 and FIG. 6 as percent reflectance.

TABLE 10
Reflectance of Exemplary Films Including 2 Weight
% TiO₂ and 2 Weight % Exemplary 1000 nm Particles,
and Comparative Films Including 4 Weight % TiO₂

| Time | C7 | E8 |
|---|---|---|
| (min up to 120) | (% ref) | (% ref) |
| 1 | 55.3 | 46.7 |
| 15 | 54.6 | 45.8 |
| 30 | 53.5 | 45.1 |
| 45 | 52.3 | 43.9 |
| 60 | 50.4 | 42.3 |
| 75 | 46.7 | 39.7 |
| 90 | 37.5 | 34.3 |
| 105 | 35.2 | 42.2 |
| 120 | 35.0 | 42.1 |
| 4 hrs | 34.8 | 42.0 |
| 24 hrs | 34.7 | 41.9 |

The results demonstrate that, when used alone, 4% TiO₂ delivers initial whitening performance, but fades upon drying, presumably due to TiO₂ agglomeration, and that the exemplary whitening formulations prepared in accordance with the present invention provide a constant and prolonged reflectance value significantly higher than comparative formulations.

What is claimed is:

1. A personal care composition comprising:
(A) voided latex particles comprising
(i) at least one core polymer comprising polymerized units derived from (a) 20 to 60 weight % of monoethylenically unsaturated monomers containing at least one carboxylic acid group, based on the total weight of the core polymer, and (b) 40 to 80 weight % of non-ionic ethylenically unsaturated monomers, based on the total weight of the core polymer; and
(ii) at least one shell polymer comprising polymerized units derived from (a) 55 to 85 weight % of non-ionic ethylenically unsaturated monomers, based on the total weight of the shell polymer(s), and (b) 15 to 45 weight % of polyethylenically unsaturated monomers, based on the total weight of the shell polymer(s); and
(B) at least one pigment grade inorganic metal oxide particle, wherein the voided latex particles are present in an amount of from 0.5 to 10 weight %, based on the total weight of the composition, and wherein the voided latex particles contain a void and have a particle size of from 400 nm to 1500 nm.

2. The personal care composition of claim 1, wherein
the non-ionic ethylenically unsaturated monomers of the at least one shell polymer comprise a monomer selected from the group consisting methyl acrylate, methyl methacrylate, ethyl acrylate, ethyl methacrylate, butyl acrylate, butyl methacrylate, isobutyl acrylate, isobutyl methacrylate, hydroxyethyl methacrylate, hydroxypropyl methacrylate, dimethylaminoethyl methacrylate, dimethylaminopropyl methacrylamide, styrene, ethylvinylbenzene, t-butylstrene, and mixtures thereof, and
the polyethylenically unsaturated monomers of the at least one shell polymer comprise a monomer selected from the group consisting of di(meth)acrylates, tri(meth)acrylates, tetra(meth)acrylates, polallylic monomers, polyvinylic monomers, (meth)acrylic monomers having mixed ethylenic functionality, and mixtures thereof.

3. The personal care composition of claim 1, wherein
the monoethylenically unsaturated monomers containing at least one carboxylic acid group of the core polymer comprise a monomer selected from the group consisting of (meth)acrylic acid, (meth)acryloxypropionic acid, itaconic acid, aconitic acid, maleic acid, maleic anhydride, fumaric acid, cronotic acid, citraconic acid, maleic anhydride, monomethyl maleate, monomethyl fumarate, monomethyl itaconate, and mixtures thereof, and
the non-ionic ethylenically unsaturated monomers of the core polymer comprise a monomer selected from the group consisting of styrene, vinyltoluene, ethylene, vinyl acetate, vinyl chloride, vinylidene chloride acrylonitrile, (meth)acrylamide, methyl (meth)acrylate, ethyl (meth)acrylate, butyl (meth)acrylate, 2-ethylhexyl (meth)acrylate, benzyl (meth)acrylate, lauryl (meth)acrylate, oleyl (meth)acrylate, palmityl (meth)acrylate, stearyl (meth)acrylate, and mixtures thereof.

4. The personal care composition of claim 1, wherein the at least one shell polymer further comprises polymerized units derived from 0.1 to 5 weight % of a monoethylenically unsaturated monomer containing at least one carboxylic acid group.

5. The personal care composition of claim 1, wherein the at least one shell polymer further comprises polymerized units derived from 0.1 to 5 weight % of a monoethylenically unsaturated monomer containing at least one “non-carboxylic” acid group.

6. The personal care composition of claim 1, wherein the voided latex particles have a void fraction of from 1% to 70%.

7. The personal care composition of claim 1, wherein the pigment grade inorganic metal oxide particles comprise one or more inorganic metal oxides selected from the group consisting of pigment grade ZnO, pigment grade TiO₂, and combinations thereof.

8. The personal care composition of claim 1, wherein the pigment grade inorganic metal oxide particles are present in an amount of from 0.1 to 20 weight %, based on the total weight of the composition.

9. A method for lightening skin tone, comprising topically administering to the skin an effective amount of a personal care composition comprising:
(A) voided latex particles comprising
(i) at least one core polymer comprising polymerized units derived from (a) 20 to 60 weight % of monoethylenically unsaturated monomers containing at least one carboxylic acid group, based on the total weight of the core polymer, and (b) 40 to 80 weight % of non-ionic ethylenically unsaturated monomers, based on the total weight of the core polymer; and
(ii) at least one shell polymer comprising polymerized units derived from (a) 55 to 85 weight % of non-ionic ethylenically unsaturated monomers, based on the total weight of the shell polymer(s), and (b) 15 to 45 weight % of polyethylenically unsaturated monomers, based on the total weight of the shell polymer(s); and
(B) pigment grade inorganic metal oxide particles,
wherein the voided latex particles are present in an amount of from 0.5 to 10 weight %, based on the total weight of the composition, and wherein the voided latex particles contain a void and have a particle size of from 400 nm to 1500 nm.

10. A method for improving visible light scattering of a composition comprising adding to said composition from 0.5 to 10 weight % of voided latex particles, based on the total weight of the composition comprising:
(i) at least one core polymer comprising polymerized units derived from (a) 20 to 60 weight % of monoethylenically unsaturated monomers containing at least one carboxylic acid group, based on the total weight of the core polymer, and (b) 40 to 80 weight % of non-ionic ethylenically unsaturated monomers, based on the total weight of the core polymer; and
(ii) at least one shell polymer comprising polymerized units derived from (a) 55 to 85 weight % of non-ionic ethylenically unsaturated monomers, based on the total weight of the shell polymer(s), and (b) 15 to 45 weight % of polyethylenically unsaturated monomers, based on the total weight of the shell polymer(s),
wherein the composition comprises at least one pigment grade inorganic metal oxide particle, and wherein the voided latex particles contain a void and have a particle size of from 400 nm to 1500 nm.
